(19) United States
(12) Patent Application Publication
(10) Pub. No.: US 2013/0008854 A1
(43) Pub. Date: Jan. 10, 2013

(54) DIALYSIS MACHINE CALIBRATION

(75) Inventors: Mark Wallace, Kinver (GB); Ben Higgit, Holywood (GB); Keith Heyes, Redditch (GB)

(73) Assignee: Quanta Fluid Solutions Ltd, Warwickshire (GB)

(21) Appl. No.: 13/261,085

(22) PCT Filed: Jun. 15, 2010
(86) PCT No.: PCT/GB2010/001160

§ 371 Date: Sep. 10, 2012

(30) Foreign Application Priority Data
Jun. 15, 2009 (GB) 0910247.6

(51) Int. Cl. B01D 61/32; A61M 1/16

(52) U.S. Cl. 210/647; 210/321.6; 210/136; 210/646

(57) ABSTRACT

The invention covers a cartridge for use in a hemodialysis machine, the cartridge comprising a dialysate flow path including a dialyzer, the dialysate flow path for delivering a flow of dialysate through the dialyzer; a mixing pump defining a chamber having volume variable between a maximum volume and a minimum volume for receiving a predetermined volume of a first dialysate solution base, second dialysate solution base, and a volume of water; a first dialysate solution base supply conduit having a first positive displacement pump having a first inlet valve and a first outlet valve associated therewith; a second dialysate solution base supply conduit having a second positive displacement pump having a second inlet valve and a second outlet valve associated therewith; a first and second fluid conduit associated joining respective first and second positive displacement pumps with the mixing pump; and a third fluid conduit for connecting the mixing pump outlet to a dialyzer filter inlet.

The present invention relates to dialysis machines and in particular, but not exclusively, to a disposable cartridge for use in hemodialysis machine.

Dialysis is a treatment which replaces the renal function of removing excess fluid and waste products, such as potassium and urea, from blood. The treatment is either employed when renal function has deteriorated to an extent that uremic syndrome becomes a threat to the body's physiology (acute renal failure) or, when a longstanding renal condition impairs the performance of the kidneys (chronic renal failure).

There are two major types of dialysis, namely hemodialysis and peritoneal dialysis.

In peritoneal dialysis treatment, a dialysate solution is run through a tube into the peritoneal cavity. The fluid is left in the cavity for a period of time in order to absorb the waste products, and is subsequently removed through the tube for disposal.

It is common for patients in the early stages of treatment for a longstanding renal condition to be treated by peritoneal dialysis before progressing to hemodialysis at a later stage.

In hemodialysis, the patient's blood is removed from the body by an arterial line, is treated by the dialysis machine, and is then returned to the body by a venous line. The machine passes the blood through a dialyser containing tubes formed from a semi-permeable membrane. On the exterior of the semi-permeable membrane is a dialysate solution. The semi-permeable membrane filters the waste products and excess fluid from the blood into the dialysate solution. The membrane allows the waste and a controlled volume of fluid to permeate into the dialysate whilst preventing the loss of larger -more desirable molecules, like blood cells and certain proteins and polypeptides.

The action of dialysis across the membrane is achieved primarily by a combination of diffusion (the migration of molecules by random motion from a region of higher concentration to a region of lower concentration), and convection (solute movement that results from bulk movement of solvent, usually in response to differences in hydrostatic pressure).

Fluid removal (otherwise known as ultrafiltration) is -achieved by altering the hydrostatic pressure of the dialysate side of the membrane, causing free water to move across the membrane along the pressure gradient.

The correction of uremic acidosis of the blood is achieved by use of a bicarbonate buffer. The bicarbonate buffer also allows the correction of the blood bicarbonate level.

The dialysis solution consists of a sterilized solution of mineral ions. These ions are contained within an acid buffer which is mixed with water and bicarbonate base prior to delivery to the dialyser. The water used is cleaned to a sufficient degree that it is suitable for use as a base for trans-membrane ion transfer with the blood (hereinafter sterile water), this may for example be achieved by known methods including reverse osmosis, heat treatment, filtration or a combination of such known methods.

Dialysate composition is critical to successful dialysis treatment since the level of dialytic exchange across the membrane, and thus the possibility to restore adequate body electrolytic concentrations and acid-base equilibrium, depends on the composition.

The correct composition is accomplished primarily by formulating a dialysate whose constituent concentrations are set to approximate normal values in the body.

However, achieving the correct composition of dialysate requires the accurate control of low volumes of liquid and at present this is achieved by the provision of complex fluid paths, including multiple pumping and valving components on the dialysis machine.

This presents the disadvantage of a complex and costly dialysis machine which is at increased risk of failure by virtue of its complexity. Increased maintenance is also a problem since it is essential to minimise machine downtime in order to most efficiently treat the patient. In addition the complexity of these systems within the machine results in overly large machines that cannot be easily transported or kept, in an unobtrusive manner, in the home.

A further problem with known hemodialysis machines is that the blood and dialysate solution lines require careful mounting onto the dialysis machine before the treatment can commence. This presents a risk that the lines are not correctly installed, a risk which is particularly relevant to those patients who dialyse at home.

This method of dialysis also presents an increased risk of cross-infection between patients since the disposable blood and dialysate lines come into contact with the dialysis machine which needs frequent and thorough cleaning between uses.

The problems associated with conventional dialysis equipment are mitigated to some degree by the system disclosed in WO 2006/120415 which discloses a cartridge based system for conducting hemodialysis, however the method and system for mixing the dialysate proposed in this application is complex and costly involving a large cartridge with multiple reservoirs, each having level control and therefore requiring a complex pumping and control system. Both this complexity and this space requirement are undesirable in portable dialysis machines, for example those suitable for home dialysis.

It is an object of the present invention to provide a hemodialysis system which at least mitigates some of the problems described above.

According to a first aspect of the invention there is provided a cartridge for use in a hemodialysis machine, the cartridge comprising:
a dialysate flow path including a dialyser, the dialysate flow path for delivering a flow of dialysate through the dialyser;
a mixing pump defining a chamber having volume variable between a maximum-volume and a minimum volume for receiving a predetermined volume of a first dialysate solution base, second dialysate solution base, and a volume of water;
a first dialysate solution base supply conduit having a first positive displacement pump having a first inlet valve and a first outlet valve associated therewith;
a second dialysate solution base supply conduit having a second positive displacement pump having a second inlet valve and a second outlet valve associated therewith;
a first and second fluid conduit associated joining respective first and second positive displacement pumps with the mixing pump; and
a third fluid conduit for connecting the mixing pump outlet to a dialyser filter inlet.

By using this apparatus an accurately mixed dialysate can be produced using a minimum of pump chambers and sensors.

Preferably the cartridge comprises a blood flow path for carrying a volume of blood to be treated in the dialyser.

Preferably the cartridge is disposable.

Preferably said first and second conduits each have a sensor therein to generate a signal indicative of the concentration of said first and second dialysate solution base flowing through said conduits.

Preferably the cartridge further comprises a sensor in the third conduit to generate a signal indicative of the concentration of the fluid flowing therethrough.

In a preferred arrangement the first and second fluid conduits feed directly into the mixing pump chamber. In an alternative preferred arrangement the first and second fluid conduits feed into a water conduit immediately upstream of the chamber of the mixing pump.

Preferably the cartridge further comprises:
a diverter valve in the third conduit, downstream of the sensor therein, having a first position in which fluid passing therethrough is directed to the dialyser and a second position in which fluid passing therethrough is diverted to a drain.

The diverter valve enables the flow path out of the mixing chamber to be directed to either a dialyser or to the drain, thus avoiding flowing out of specification dialysate through the dialyser.

Preferably the first inlet valve and first outlet valve, and the second inlet valve and second outlet valve comprise first and second anti siphon check valves, each having three ports and two flow paths therethrough, one connecting the dialysate solution base supply conduit to the associated positive displacement and the second connecting the associated positive displacement pump to the mixing pump chamber.

According to a second aspect of the invention there is provided a method of calibrating a dialysate mixing pump for mixing a tri-mix dialysate comprising a mixture of first dialysate base solution, second dialysate base solution and water in specific ratios, said method comprising the steps of:
a) predicting the required volume of a first dialysate solution base needed to create a dialysate having a required concentration of said first dialysate solution base;
b) operating a first dialysate solution base pump to add said predicted volume of first dialysate solution base to a volume of water in a mixing pump and mixing said first dialysate solution base with said water therein to form a first mixture;
c) expelling said first mixture from the mixing pump and measuring a parameter indicative of the concentration of said first mixture;
d) modifying the first dialysate solution base pump controls to increase or decrease the amount of first dialysate solution base added to said mixing pump dependant on the measured a parameter;
e) predicting the required volume of a second dialysate solution base needed to create a dialysate having a required concentration of said second dialysate solution base;
f) operating a second dialysate solution base pump to add said predicted volume of second dialysate solution base to a volume of water in a mixing pump and mixing said second dialysate solution base with said water therein to form a second mixture;
g) expelling said second mixture from the mixing pump and measuring a parameter indicative of the concentration of said second mixture;
h) modifying the second dialysate solution base pump controls to increase or decrease the amount of second dialysate solution base added to said mixing pump dependant said parameter;
i) operating the first and second dialysate solution base pumps to add the modified volume of first and second dialysate solution bases to a volume of water in the mixing pump chamber to achieve a required tri-mix of first dialysate base solution, second dialysate base solution and water.

By this method the apparatus having a minimum number of mixing chambers and sensors can be calibrated to produce an accurately mixed dialysate.

Preferably the method further comprises the step of measuring a parameter indicative of the concentration of the first dialysate solution base and using said measurement in the prediction of the required volume of the first dialysate solution base needed.

Preferably the method further comprises the step of measuring a parameter indicative of the concentration of the second dialysate solution base and using said measurement in the prediction of the required volume of the second dialysate solution base needed.

Preferably after step h) the method further comprises the step of repeating steps e) to h) until the concentration of the first mixture is equal to the required concentration of the first dialysate solution base.

Preferably the method further comprises the step of: once the required concentration is achieved, recording a parameter indicative of the volume of first dialysate solution base added to said mixing pump to create a first mixture having the required concentration.

Preferably after step d) the method further comprises the step of repeating steps a) to d) until the concentration of the second mixture is equal to the required concentration of the second dialysate solution base.

Preferably the method further comprises the step of: once the required concentration is achieved, recording a parameter indicative of the volume of second dialysate solution base added to said mixing pump to create a second mixture having the required concentration.

Preferably the method further comprises: measuring a parameter indicative of the concentration of the tri-mix to verify it has the correct concentration. Preferably the method further comprises the step of: if the measured parameter indicates the tri-mix does not have the correct concentration, repeating the calibration.

Preferably the method further comprises monitoring a parameter indicative of the concentration of the first dialysate solution base.

Preferably the method further comprises the step of: if the measured parameter indicates first dialysate solution base changes concentration beyond a specified range, repeating the steps of claim 8.

Preferably the method further comprises the step of: monitoring a parameter indicative of the concentration of the second dialysate solution base. Preferably the method further comprises the step of: if the measured parameter indicates second dialysate solution base changes concentration beyond a specified range, repeating the calibration.

Preferably the method further comprises: if the mixture of the tri mix dialysate is outside of its specified ratios, diverting the flow of dialysate to drain.

Preferably the method further comprises: if the mixture of the tri mix dialysate is within its specified ratios, diverting the flow of dialysate to a dialyser.

The invention will now be described, by way of example only, and with reference to the following drawings, in which:

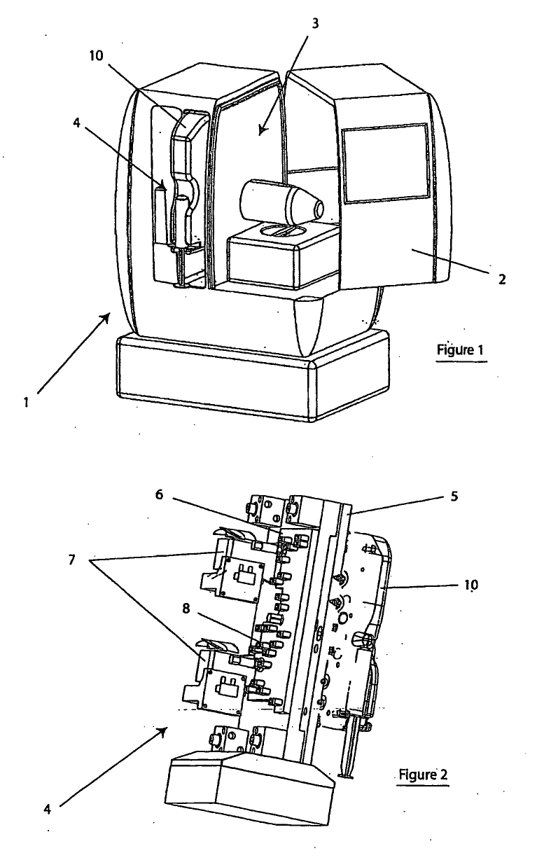

FIG. 1 is an isometric view of the dialysis machine for use with the cartridge of the current invention,

FIG. 2 is an isometric view of the engine portion of the machine of FIG. 1,

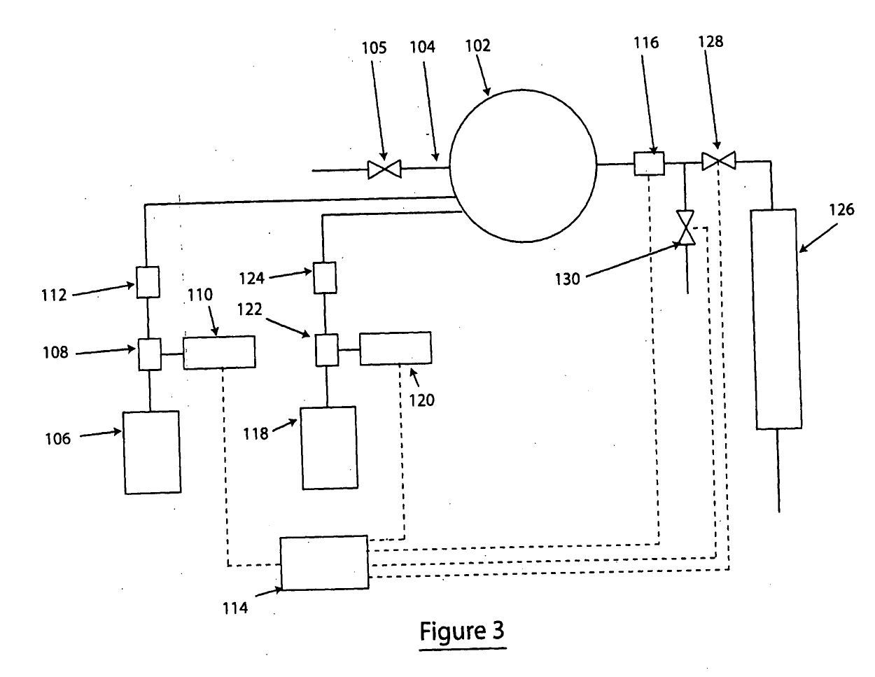

FIG. 3 is a schematic diagram of the dialysate flow path for use on a cartridge of the invention; and

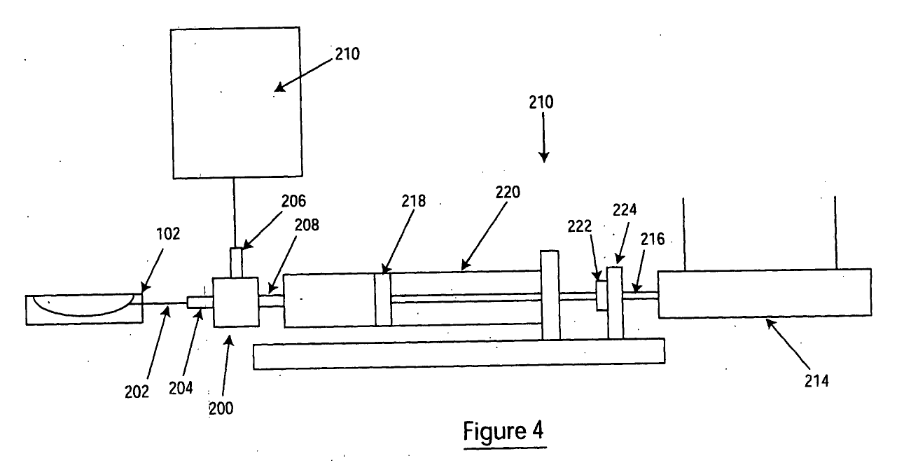

FIG. 4 is a schematic representation of a dialysate solution base delivery system according to the present invention.

In FIG. 1 a dialysis machine 1 is shown having a cover 2 which opens to reveal a storage compartment 3. The machine has an engine section 4 which receives a dialysis cartridge 10.

Referring now to FIG. 2, the engine section 4 is shown in further detail to include first and second platens 5, 6 which close upon insertion of the cartridge 10 into the machine to retain the cartridge in position in use. The engine 4 has pneumatic actuators 7 and sensors (indicated generally at 8 in FIG. 2) arranged on the second platen to control operation of the cartridge 10 as will be described in further detail shortly.

In general the dialysate cartridge may have all the fluid flow paths and functions for the supply of dialysate and blood to a dialyser incorporated therein, or may purely be a cartridge for the preparation of dialysate. The cartridge comprises a moulded plastics cartridge having fluid flow paths defined in the surface thereof. At least one surface of the cartridge is covered by a flexible membrane, formed from a deformable plastics material, enclosing the fluid flow paths. The cartridge has a number of inlets for clean water, first dialysate solution base and second dialysate solution base, and an outlet to drain. The cartridge may also have an outlet for the mixed tri-mix dialysate from which it can be supplied to a dialyser, or alternatively the cartridge may contain all the flow paths necessary for dialysate and blood, and a dialyser filter, onboard such that the entire dialysis process can take place on the cartridge. Further details of how such a cartridge may be constructed can be found in WO 2006/120415.

Referring to FIG. 3 a schematic diagram is shown of a dialysate preparation flow path suitable for incorporation onto a cartridge for use in the machine of FIG. 1. A mixing pump having a chamber 102 is supplied with a supply of clean water via conduit 104 and valve 105. Prior to supply to the chamber the clean water will have undergone treatment, for example reverse osmosis, to clean it and remove any bacteria to a level acceptable for use in a hemodialysis process. A saturated solution of bicarbonate 106 is prepared remotely from the cartridge 100 and fed to a three way valve 108 of a bicarbonate pump 110. A controller 114, having been inputted, either manually or electronically, with a required concentration of bicarbonate in the tri mix, makes an initial calculation of the volume of bicarbonate to be added to the chamber 102 of the mixing pump of a known volume, based on the temperature and operates the bicarbonate pump 110 to add that volume to the chamber 102, via the three way valve 108. The bicarbonate is added either before and/or simultaneously to the water entering the chamber 102 of the mixing pump. Fluid turbulence within the chamber 102 of the mixing pump, both as water is drawn in, and as the mixture is expelled causes the bicarbonate and water to mix thoroughly. As the concentration of the saturated bicarbonate solution can change with temperature and conditions, and as there may be manufacturing tolerance differences in the exact volume of the chamber 102 from one cartridge 100 to another it is virtually impossible to get the exact mixture required, purely by predicting the required volume of bicarbonate needed to be added by the bicarbonate pump 110.

A first conductivity sensor 112 measures the conductivity of the saturated bicarbonate solution to create a signal indicative of its concentration, and feeds this signal back to the controller 114. The controller 114 can then increase or decrease the amount of bicarbonate added by the bicarbonate pump 110 to compensate for any variance between the predicted concentration of the saturated bicarbonate solution and the actual concentration of the bicarbonate solution.

A second conductivity sensor 116 is placed downstream of the pump chamber 102 and measures the conductivity of the fluid exiting the mixing pump chamber 102 to create a signal indicative of the concentration of the mixture being pumped from the chamber 102 and feed it back to the controller 114. The controller 114 can then increase or decrease the amount of bicarbonate added by the bicarbonate pump 110 to achieve the required concentration of bicarbonate in the bicarbonate water mixture generated in the mixing pump.

The process is repeated until the required concentration of bicarbonate and water is achieved. When this is achieved, control parameters for controlling the bicarbonate pump 110 are stored in the controller 114.

The process is then repeated but with acid solution from a supply 118 via an acid solution pump 120 and associated dual check valve 122 and conductivity sensor 124 until the required concentration of acid solution and water has been established. Again signals are fed back to the controller 114 indicative of the concentration of the acid solution being added to the mixing pump chamber 102 and indicative of the concentration of water/acid solution mixture generated in the mixing pump chamber 102, the controller modifying the control of the acid solution pump 120 in response to these signals to achieve the required mixture of acid solution and water. When the required concentration of acid solution and water is achieved, control parameters for controlling the acid solution pump 120 are stored in the controller.

Once the pump control parameters for the bicarbonate pump 110 and the acid solution pump 120 have been independently established, both bicarbonate pump 110 and acid pump 120 are operated simultaneously by the controller to add bicarbonate solution and acid solution respectively to the mixing pump chamber 102 so as to mix with water therein. The tri-mixture dialysate of bicarbonate solution, acid solution and water is then expelled from the chamber 102 and its conductivity is measured by conductivity sensor 116 to verify that it is of the correct concentration. The tri-mixture dialysate can then be passed through a dialyser 126, which may be a part of the cartridge, attached thereto or remote therefrom, and used to dialyze blood. Prior to use, i.e. during the calibration routine, the fluid emitting from the mixing pump chamber is drained. The drain path may flow through the dialyser, i.e. the same path as used in treatment, or alternatively a separate drain path may be provided leading from the conduit joining the mixing pump chamber 102 and the dialyser 126 to a drain point. In this arrangement, valves 128, 130 control the flow of the dialysate to either pass through the dialyser or to flow directly to the drain. In use, if, during the dialysis process, the conductivity sensor 116 detects that the concentration of the tri-mix dialysate has gone out of specification the controller 114 actuates valves 128, 130 to divert the flow of the dialysate to the drain, bypassing the filter, and the calibration process is repeated. Once the pumps and control are again producing dialysate within specification the controller 114 actuates valves 128, 130 again such that the dialysate flows through the dialyser and the process continues.

In addition to the functions described above the sensors 112, 122 can be used as safety sensors and will detect a change of fluid from dialysate solution base to air and thus can detect if the source of dialysate solution base 106, 118 runs out or if air becomes drawn into the system at that point. The flow can then be stopped accordingly to prevent air from entering the dialyser filter 126.

Referring now to FIG. 4, the positive pump and three-way valve on the current invention are shown schematically in further detail. The three-way valve is indicated generally at 200.

The mixing pump chamber 102 is connected via a fluid line 202 to an output 204 of the three-way valve 106. The three-way valve also has a reservoir inlet 206 and a pump inlet 208. The reservoir inlet 206 is connected to a reservoir 210 containing the acid or bicarbonate solution. The reservoir 210 is provided on the dialysis machine, or attached thereto, and does not form part of the cartridge itself. The positive displacement pump is indicated generally at 212. The positive displacement pump includes a pneumatic cylinder 214 which drives a piston arm 216 in a reciprocating manner. At the opposite end of the piston arm 216 to the piston cylinder 214 is a plunger 218 which acts within a pump chamber 220 integral within the cartridge.

On the return stroke indicated at A in FIG. 4, the plunger 219 is moved within the chamber 220 to draw in to the chamber 220 a measured volume of fluid from the bicarbonate/acid solution reservoir 210. This transfer of fluid is achieved by the closure of the three-way valve output 204, with the reservoir inlet 206 and pump inlet 208 remaining open. The piston arm 216 is withdrawn in direction A until an abutment 222 provided on the piston arm 216 comes into contact with a moveable end stop 224.

Upon the abutment 222 hitting the moveable end stop 224, the pneumatic cylinder 214 is driven in direction B in order to dispense the dialysate solution from the chamber 220 into the mixing pump chamber 102. This transfer of fluids is achieved by the closure of the reservoir inlet 206, and the opening of the three-way valve output 204. The pneumatic cylinder 214 drives the plunger 218 in direction B until the plunger abuts the extreme left hand end of the chamber 220. Accordingly, by reciprocating the movement of the cylinder piston aim 216 in a known manner, a quantity of bicarbonate/acid solution is repeatedly dispensed into the mixing pump chamber 102. Furthermore, by adjusting the position of the removable end stop 224, the volume of fluid dispensed can be accurately set. The moveable end stop 224 is positioned by a stepper motor or similar accurate positioning drive system.

It will be appreciated that the above description is given by way of example only and it is anticipated that various changes may be made to the specific arrangement of components without departing from the scope of the invention, for example the pumping arrangement may be different from that described in relation to FIG. 4.

1. A cartridge for use in a hemodialysis machine, the cartridge comprising:
a dialysate flow path, including a dialyser, the dialysate flow path for delivering a flow of dialysate through the dialyser;
a mixing pump defining a chamber having volume variable between a maximum volume and a minimum volume for receiving a predetermined volume of a first dialysate solution base, second dialysate solution base, and a volume of water;
a first dialysate solution base supply conduit having a first positive displacement pump having a first inlet valve and a first outlet valve associated therewith;
a second dialysate solution base supply conduit having a second positive displacement pump having a second inlet valve and a second outlet valve associated therewith;
a first and second fluid conduit associated joining respective first and second positive displacement pumps with the mixing pump; and
a third fluid conduit for connecting the mixing pump outlet to a dialyser filter inlet.

2. A cartridge according to claim 1 wherein said first and second conduits each have a sensor therein to generate a signal indicative of the concentration of said first and second dialysate solution base flowing through said conduits.

3. A cartridge according to claim 1 further comprising a sensor in the third conduit to generate a signal indicative of the concentration of the fluid flowing therethrough.

4. A cartridge according to claim 1 wherein the first and second fluid conduits feed directly into the mixing pump chamber.

5. A cartridge according to claim 1 wherein the first and second fluid conduits feed into a water conduit immediately upstream of the chamber of the mixing pump.

6. A cartridge according to claim 3 further comprising:
a diverter valve in the third conduit, downstream of the sensor therein, having a first position in which fluid passing therethrough is directed to the dialyser and a second position in which fluid passing therethrough is diverted to a drain.

7. A cartridge according to claim 1 wherein the first inlet valve and first outlet valve, and the second inlet valve and second outlet valve comprise first and second anti siphon check valves, each having three ports and two flow paths therethrough, one connecting the dialysate solution base supply conduit to the associated positive displacement and the second connecting the associated positive displacement pump to the mixing pump chamber.

8. A method of calibrating a dialysate mixing pump for mixing a tri-mix dialysate comprising a mixture of first dialysate base solution, second dialysate base solution and water in specific ratios, said method comprising the steps of:
a) predicting the required volume of a first dialysate solution base needed to create a dialysate having a required concentration of said first dialysate solution base;
b) operating a first dialysate solution base pump to add said predicted volume of first dialysate solution base to a volume of water in a mixing pump and mixing said first dialysate solution base with said water therein to form a first mixture;
c) expelling said first mixture from the mixing pump and measuring a parameter indicative of the concentration of said first mixture;
d) modifying the first dialysate solution base pump controls to increase or decrease the amount of first dialysate solution base added to said mixing pump dependant on the measured a parameter;
e) predicting the required volume of a second dialysate solution base needed to create a dialysate having a required concentration of said second dialysate solution base;
f) operating a second dialysate solution base pump to add said predicted volume of second dialysate solution base to a volume of water in a mixing pump and mixing said second dialysate solution base with said water therein to form a second mixture;
g) expelling said second mixture from the mixing pump and measuring a parameter indicative of the concentration of said second mixture;
h) modifying the second dialysate solution base pump controls to increase or decrease the amount of second dialysate solution base added to said mixing pump dependant said parameter;
i) operating the first and second dialysate solution base pumps to add the modified volume of first and second dialysate solution bases to a volume of water in the mixing pump chamber to achieve a required tri-mix of first dialysate base solution, second dialysate base solution and water.

9. The method according to claim 8 further comprising the step of measuring a parameter indicative of the concentration of the first dialysate solution base and using said measurement in the prediction of the required volume of the first dialysate solution base needed.

10. The method according to claim 8 further comprising the step of measuring a parameter indicative of the concentration of the second dialysate solution base and using said measurement in the prediction of the required volume of the second dialysate solution base needed.

11. The method according to claim 8 wherein after step h) the method further comprises the step of repeating steps e) to h) until the concentration of the first mixture is equal to the required concentration of the first dialysate solution base.

12. The method according to claim 11 further comprising the step of:
once the required concentration is achieved, recording a parameter indicative of the volume of first dialysate solution base added to said mixing pump to create a first mixture having the required concentration.

13. The method according to claim 8 wherein after step d) the method further comprises the step of repeating steps a) to d) until the concentration of the second mixture is equal to the required concentration of the second dialysate solution base.

14. The method according to claim 13 further comprising the step of:
once the required concentration is achieved, recording a parameter indicative of the volume of second dialysate solution base added to said mixing pump to create a second mixture having the required concentration.

15. The method according to claim 8 further comprising:
measuring a parameter indicative of the concentration of the tri-mix to verify it has the correct concentration.

16. The method according to claim 15 further comprising:
if the measured parameter indicates the tri-mix does not have the correct concentration, repeating the steps of claim 8.

17. The method according to claim 15 further comprising:
monitoring a parameter indicative of the concentration of the first dialysate solution base.

18. The method according to claim 15 further comprising:
if the measured parameter indicates first dialysate solution base changes concentration beyond a specified range, repeating the steps of claim 8.

19. The method according to claim 8 further comprising:
monitoring a parameter indicative of the concentration of the second dialysate solution base.

20. The method according to claim 19 further comprising:
if the measured parameter indicates second dialysate solution base changes concentration beyond a specified range, repeating the steps of claim 8.

21. The method according to claim 8 further comprising:
if the mixture of the tri mix dialysate is outside of its specified ratios, diverting the flow of dialysate to drain.

22. The method according to claim 8 further comprising:
if the mixture of the tri mix dialysate is within its specified ratios, diverting the flow of dialysate to a dialyser.

23. A cartridge according to claim 1 wherein the cartridge includes a blood flow path for carrying a volume of blood to be treated in the dialyser.

24. A cartridge according to claim 1 wherein the cartridge is disposable.
